(19) United States
(12) Patent Application Publication
(10) Pub. No.: US 2010/0087753 A1
(43) Pub. Date: Apr. 8, 2010

(54) LANCING DEVICE FOR TAKING BLOOD FOR MEDICAL TESTS

(75) Inventor: Robert Wessel, Regensburg (DE)

Correspondence Address: BLACK LOWE & GRAHAM, PLLC, 701 FIFTH AVENUE, SUITE 4800, SEATTLE, WA, 98104, US

(73) Assignee: GERRESHEIMER WILDEN GMBH, Regensburg (DE)

(21) Appl. No.: 12/529,896

(22) PCT Filed: Feb. 29, 2008
(86) PCT No.: PCT/EP2008/052481

§ 371 Date: Oct. 29, 2009

(30) Foreign Application Priority Data
Mar. 5, 2007 (DE) 10 2007 011 002.4
May 24, 2007 (DE) 10 2007 024 173.0

(51) Int. Cl. A61B 5/151

(52) U.S. Cl. 600/583

(57) ABSTRACT

A lancing device for taking blood for medical examinations, comprising a base body, at least one needle arranged therein which can be extended outwards with a tip end, having a needle holding element which at least partially encompasses the needle, and a manual actuating element for releasing a displacement movement of the needle together with the needle holding element relative to the base body, wherein a movable plastic spring element for generating a pretension by means of the manual actuating element, which is connected to a control curve section that can be moved in the base body, is arranged in at least one component in the base body, wherein the needle holding element is connected to the control curve section and can automatically be moved in the forward and backward direction during a pressure release of the plastic spring element by travelling along the control curve section.

The invention relates to a lancing device for taking blood for medical examinations, comprising a base body (main body), at least one needle which is arranged therein and which can be extended outwards with a tip end, having a needle holding element which at least partially encompasses the needle, and a manual actuating element for releasing a displacement movement of the needle together with the needle holding element relative to the base body (main body), according to the preamble of claim 1.

Lancing devices of various types are known. By way of example, DE 196 17 000 C1 discloses a device comprising a needle which serves for taking blood, wherein a cannula shaped like a hollow needle is designed with a sharpened cutting edge. Such devices are usually not intended for single use and, due to the needle shape, are not always pain-free during use.

A system for taking blood comprising a blood receiving cylinder with a piston and a needle cylinder head with a needle holder which can be screwed onto an outer thread of the blood receiving cylinder is known from DE 44 43 276 A1. Such a system for taking blood is expressly intended for reuse and is designed so as to allow a germ-free reuse of this system. Accordingly, such systems are designed in an expensive manner and nevertheless harbour the risk of infections.

DE 297 18 679 U1 discloses a lancet for taking blood with a needle tip end protruding at the end region. The needle has an end region with the tip end which protrudes out from an axial end of a base body (main body) and which is embedded in a tubular attachment or connection region of a needle insertion head.

The insertion head and the base body (main body) are formed in one piece. An axial pressure is exerted on this base body (main body) by means of a device (not shown) in order to allow the needle to penetrate to a certain depth into the body of the patient. The needle end region is then manually pulled out of the body of the patient. Such devices generally do not allow a pain-free lancing operation, since the handling by means of an additional pressure-exerting device allows only the insertion but not the removal of the needle.

Accordingly, the object of the present invention is to provide a lancing device for taking blood for medical examinations, which is intended for single use, allows an almost pain-free lancing operation, is inexpensive and can even be used by the patient himself.

This object is achieved by a lancing device having the features of claim 1.

One essential point of the invention lies in the fact that, in a lancing device for taking blood for medical examinations, comprising a base body (main body), at least one needle which is arranged therein and which can be extended outwards with a tip end, having a needle holding element which at least partially encompasses the needle, and a manual actuating element for releasing a displacement movement of the needle together with the needle holding element relative to the base body (main body), a displaceable or pivotable, preferably arc-shaped or meandering, plastic spring element for generating a pretension by means of the manual actuating element is arranged in at least one component arranged in the base body (main body). This plastic spring element is connected to a control curve section (guiding curve section) that can be displaced or pivoted in the base body (main body), wherein the needle holding element is connected to the control curve section and can automatically be displaced in the forward and/or backward direction during a pressure release of the plastic spring element by travelling along the displaced or pivoted control curve section.

Due to the spring-loading of the needle holding element and thus of the needle, the latter cannot only be extended and retracted in an automatic and simple manner, so that a quick and almost pain-free lancing operation takes place, but rather, when using the spring element made from plastic, the component and thus the entire lancing device can be produced in a simple and inexpensive manner in large quantities by means of the plastic injection-moulding process. Such lancing devices are intended for single use and can be used by any person, i.e. even by untrained patients, due to the automatic extension and retraction of the needle brought about by the spring element in conjunction with the control curve section. This has the advantage that diabetics for example, who regularly have to take blood in order to check the blood sugar level, can take blood even by themselves and thus do not need to seek out a doctor or a hospital for this purpose.

The needle is to be understood to mean not only a needle-shaped element with a round cross section, but rather also a needle-shaped element with a rectangular cross section and a tip shaped as a cutting edge, which results in a cut-type wound.

According to one preferred embodiment, the lancing device comprises a tensioning curve section which is arranged on the component and runs at an angle to the direction of the displacement movement, which tensioning curve section is connected to the plastic spring element, preferably of arc-shaped design, and, during a displacement movement of the component relative to the base body (main body), can be varied in terms of its course direction by sliding along a protrusion arranged on the base body (main body). As a result, a pretension is at the same time built up in the plastic spring element as a result of the displacement of the component within the base body (main body) by means of the manual actuating element, wherein the direction of displacement of the manual actuating element and thus of the component in a first time portion is the same as the direction of displacement in a second time portion, in which the manual actuating element triggers the pressure release of the plastic spring element and the pressure in the latter is released during the forward sliding and the subsequent backward sliding of the needle out of and into the housing or the base body (main body). Alternatively, instead of the protrusion, a depressed contour or recess may be formed on the inner wall of the base body (main body), in which there engages the tensioning curve section, which is not groove-like but rather protrudes.

Such a lancing device according to this embodiment is characterised in that the displacement movements of the needle holding element and of the component have the same direction while the needle holding element is being extended outwards.

Alternatively, the displacement movements of the needle holding element and of the component may run perpendicular or at an angle to one another.

The protrusion has preferably a triangular basic shape in order to allow an optimised performance of the sliding movement between the protrusion and the tensioning curve section and in particular while a free end of the tensioning curve section slides past the protrusion since, after the free end has slid past the protrusion, an automatic return or resetting of the tensioning curve section, which is connected to the plastic spring element, is intended to take place in order to release the pressure in the plastic spring element. This represents the trigger process for the pressure release of the plastic spring element.

Preferably, the component is formed in one piece together with the control curve section, the tensioning curve section and the plastic spring element, so that an inexpensive and simple production of this entire plastic element by injection-moulding is possible.

According to a further embodiment, the plastic spring element is arranged on the manual actuating element itself, which is preferably mounted such that it can be pivoted relative to the component. By actuating the manual actuating element, the plastic spring element can be pretensioned against a further pivoting element which comprises the control curve section, wherein the pivoting element is mounted such that it can pivot relative to the component. This requires a pivoting element which may be connected in one piece or in two pieces with the component.

Alternatively, the plastic spring element may be arranged on the pivoting element, which is designed such that it can pivot relative to the manual actuating element.

The manual actuating element has a release process for displacing a retaining protrusion of the needle holding element or of the pivoting element out of its arrested position. This ensures that a displacement of the needle holding element and thus of the needle is possible only in a certain pivot position of the manual actuating element. Ideally, this start process is dependent on the current tensioned state of the plastic spring being pretensioned by the pivoting manual actuating element so that, once this release protrusion is reached, a pressure release occurs which is sufficient to extend and then retract the needle holding element at the speed necessary for a pain-free lancing operation.

Preferably, the pivotable manual actuating element and the component have a latching mechanism for latching the pivotable manual actuating element in a predefined pivot position and thus a predefined pretension of the plastic element in the component. Such a latching mechanism is intended to ensure that reuse of the disposable lancing aid according to the invention is not possible.

Ideally, a latching of the pivotable manual actuating element takes place at the same time as the triggering of a pressure release of the plastic spring element, so that the lancing aid cannot be pretensioned and a latching takes place without causing any triggering of the pressure release. This might lead to an unintended release or to a loss of pretension due to plastic creep phenomena. In any case, the latching force should be low compared to the release force, so that the latching takes place “automatically” during the release movement without the user being able to differentiate this. This latching of the actuating element is provided for lancing aids according to all the embodiments. The aim of the latching is that the lancing aid cannot be used a second time after a release operation has taken place, since the actuating part does not carry out any return movement.

In all the embodiments, both the component and the manual actuating element may be formed as two separate components or preferably in one piece.

Preferably for all the embodiments, the control curve section has a profile identical or similar to a V-shape so that, when the lowest point of the V-shaped profile is reached, the needle is in its outermost position and then is retracted again.

The control curve section is preferably designed in terms of its profile as a web or rail which is encompassed on both sides by the needle holding element so as to slide along it in the web profile direction. Alternatively, a groove may be arranged within the component or the pivoting element, which groove accordingly receives webs or protrusions of complementary shape on the needle holding element in order to allow these to slide along within the groove in the profile direction thereof, which is preferably V-shaped.

Further advantageous embodiments will emerge from the dependent claims.

Advantages and expedient features can be found in the following description in conjunction with the drawing, in which:

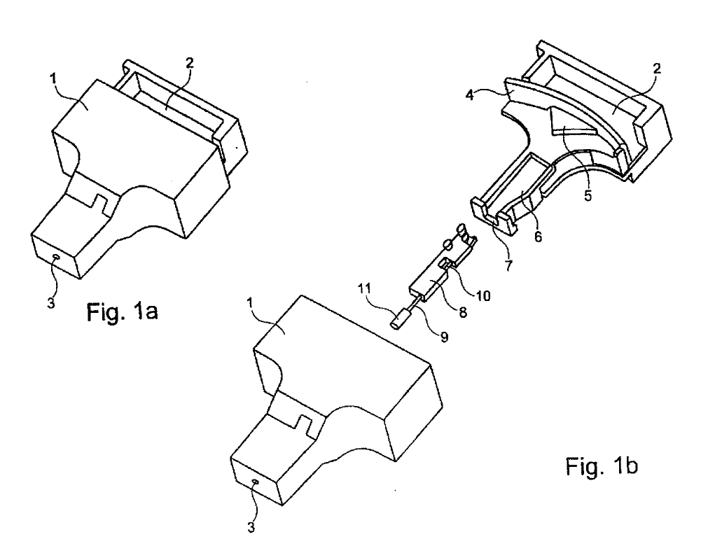

FIG. 1a shows a perspective view of a lancing device according to a first embodiment of the invention in the assembled state;

FIG. 1b shows a perspective view of a lancing device in a disassembled state;

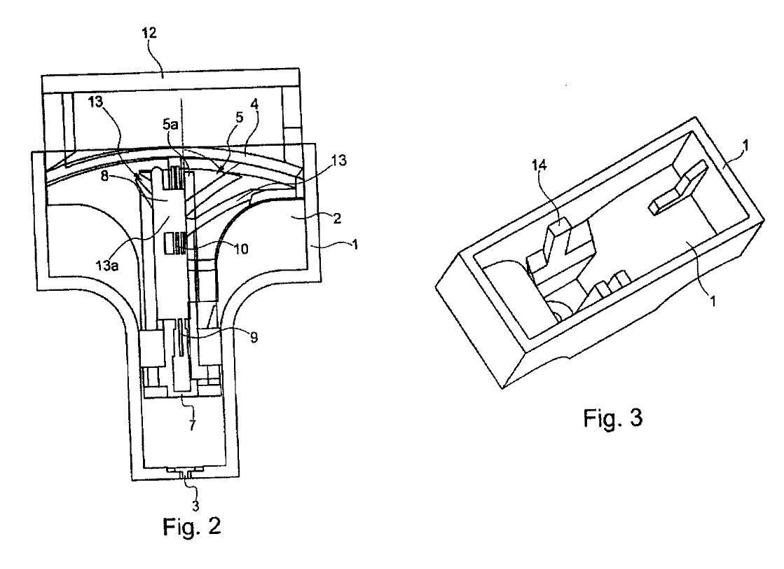

FIG. 2 shows an open and partially cross-sectional front view of the lancing device according to the first embodiment of the invention;

FIG. 3 shows a perspective plan view of the lancing device according to the first embodiment of the invention;

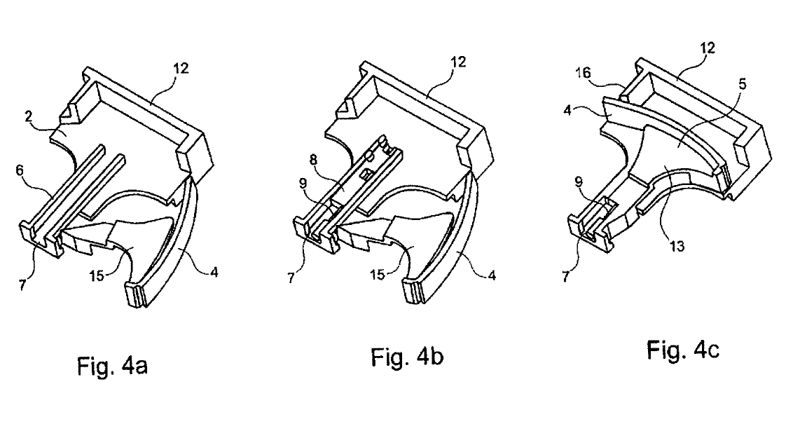

FIGS. 4a-4c show various perspective views of a component arranged in the lancing device according to the first embodiment of the invention, in individual assembly steps;

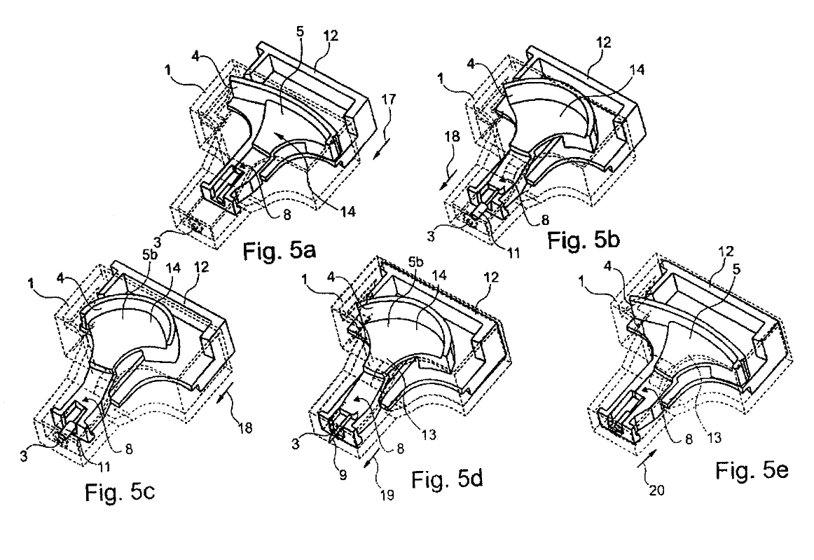

FIGS. 5a-5e show a perspective view of the lancing device according to the first embodiment of the invention in various operating states;

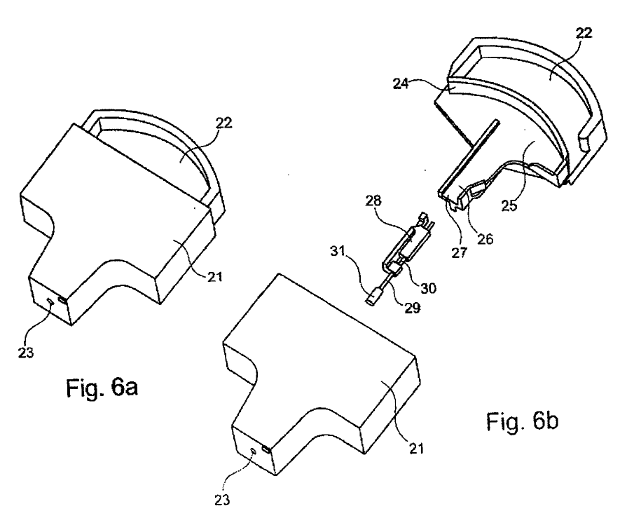

FIG. 6a shows a perspective view of a lancing device according to a second embodiment of the invention in the assembled state;

FIG. 6b shows a perspective view of the lancing device according to the second embodiment of the invention in the disassembled state;

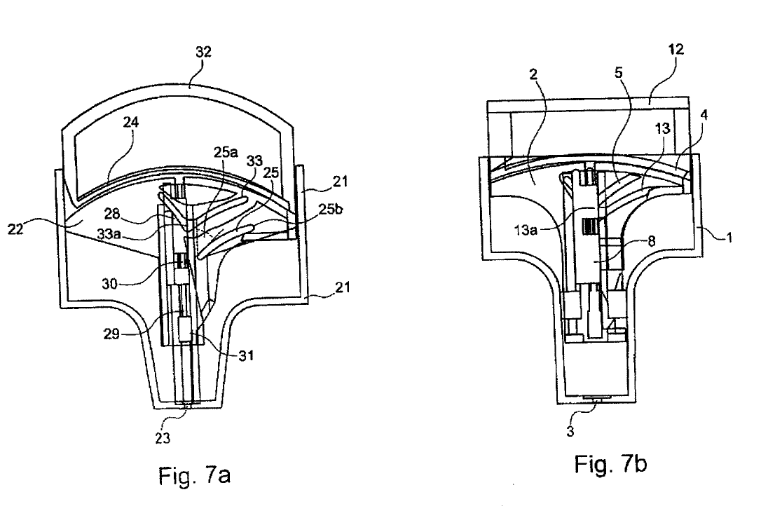

FIG. 7a shows an open and partially cross-sectional front view of the lancing device according to the second embodiment of the invention;

FIG. 7b shows an open and partially cross-sectional front view of the lancing device according to the first embodiment of the invention;

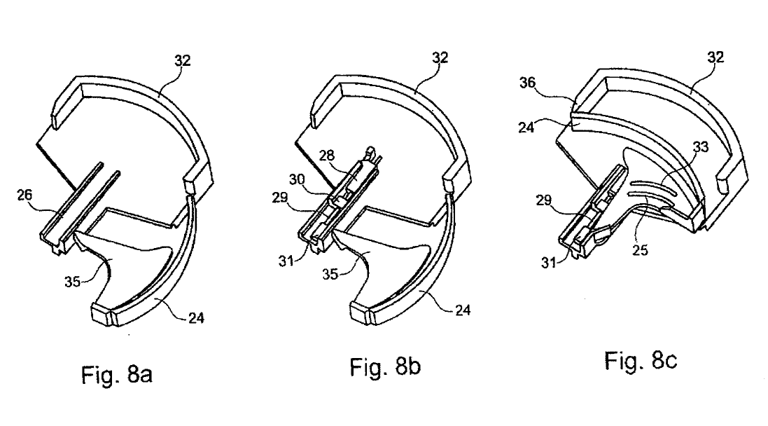

FIGS. 8a-8c show a perspective view of a component for use in a lancing device according to the second embodiment of the invention, in individual assembly steps;

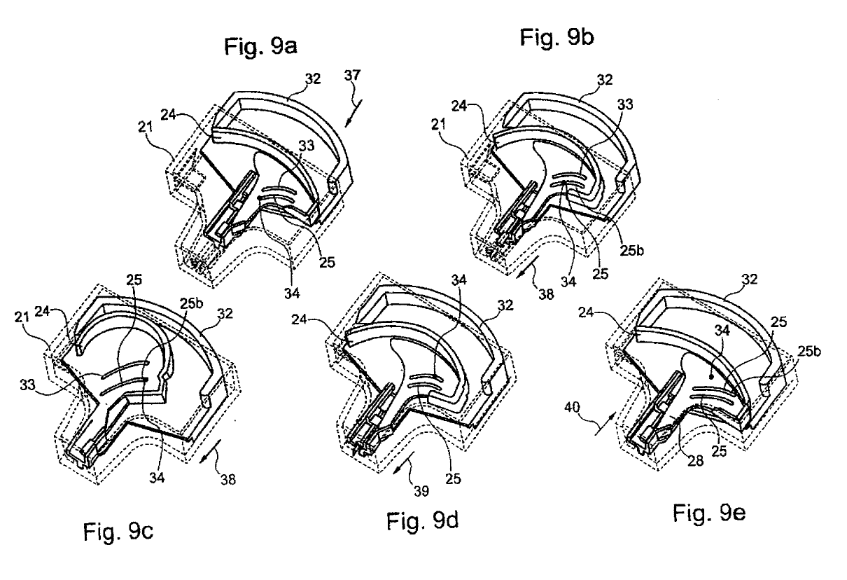

FIGS. 9a-9e show a perspective view of the lancing device according to the second embodiment of the invention in various operating states;

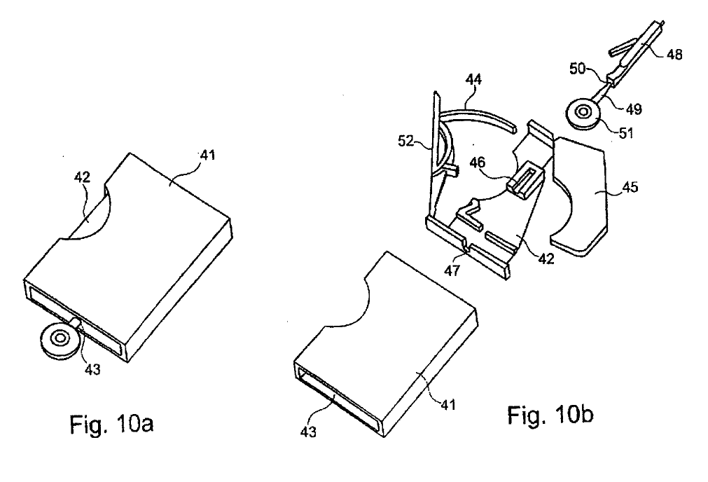

FIG. 10a shows a perspective view of the lancing device according to a third embodiment of the invention in the assembled state;

FIG. 10b shows a perspective view of the lancing device according to the third embodiment of the invention in the disassembled state;

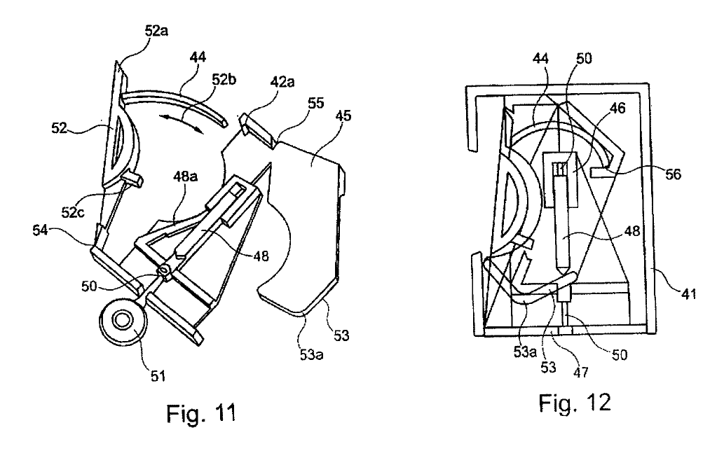

FIG. 11 shows a perspective view of the component arranged in the lancing device according to the third embodiment;

FIG. 12 shows an open and partially cross-sectional front view of the lancing device according to the third embodiment of the invention;

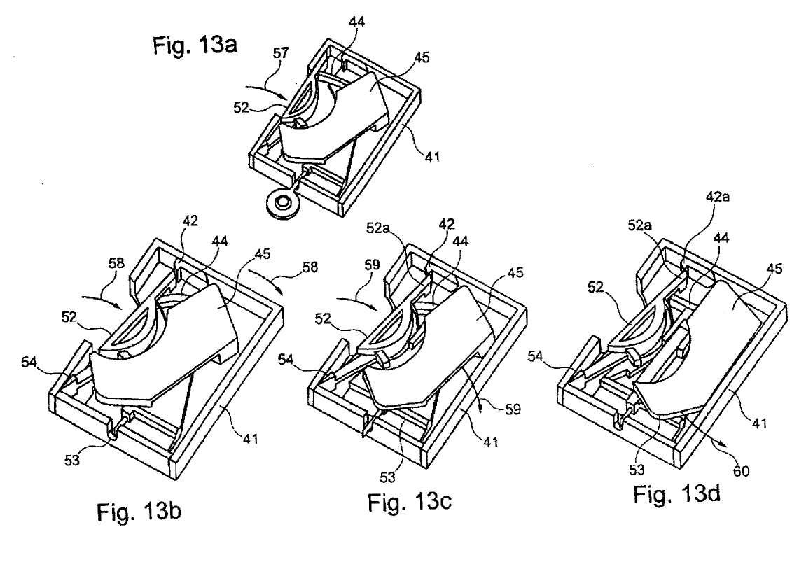

FIGS. 13a-13d show an open view of the lancing device according to the third embodiment of the invention in various operating states;

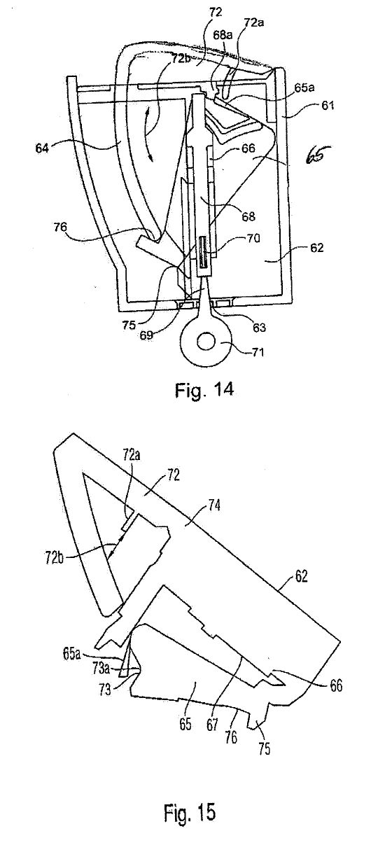

FIG. 14 shows an open and partially cross-sectional front view of the lancing device according to a fourth embodiment of the invention;

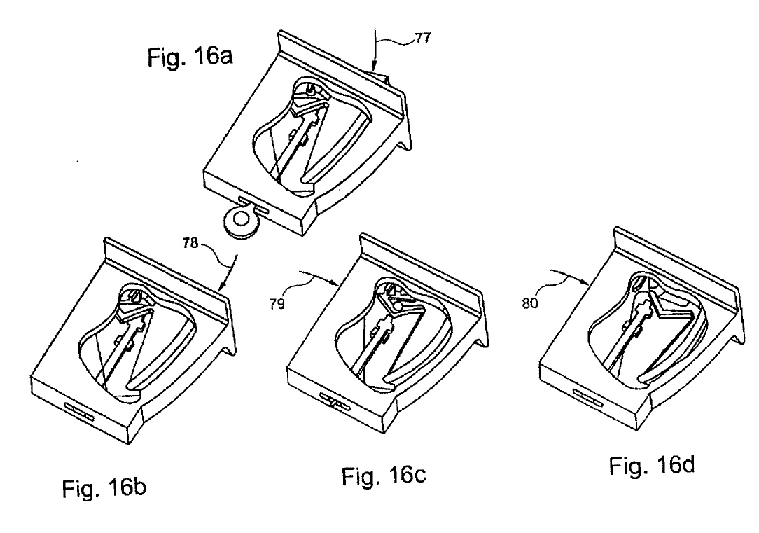

FIG. 15 shows a perspective view of a component of the lancing device according to the fourth embodiment of the invention; and

FIGS. 16a-16d show a perspective, partially open view of the lancing device according to the fourth embodiment of the invention in various operating states.

FIG. 1a shows a perspective view of a lancing device according to a first embodiment of the invention. The lancing device comprises a base body (main body) 1, which is designed as a housing, and a component 2 arranged therein which can be displaced relative to the base body (main body) 1. A needle can emerge from a lower end 3 of the base body (main body) 1.

FIG. 1b shows a perspective view of the lancing device according to the first embodiment of the invention in a disassembled state. The component 2 is removed from the base body (main body) 1 and has a plastic spring 4 or a plastic spring element 4 which is of arc-shaped design and is connected thereto by means of a tensioning curve section 5 which is arranged on the component 2 at an angle 5a to the displacement movement of a needle, as can be seen from FIG. 2.

A needle holding element 8 is arranged within a recess 6 which is oriented in the longitudinal direction of the base body (main body) 1 and of the component 2 and has a bottom opening 7, said needle holding element in turn comprising a needle 10 with an associated tip end 9 which is closed by a protective cap 11. The needle holding element 8 can be displaced forwards and backwards within the guide 6.

The protective cap 11 serves as sterile protection for the needle and is pierced during the lancing operation. It is made from an elastomer.

FIG. 2 shows an open and partially cross-sectional front view of the lancing device according to the first embodiment of the invention.

It can clearly be seen from this diagram that, as soon as a manual actuating element 12 is actuated or displaced downwards from above, a displacement of the entire component 2 in the downward direction takes place.

During such a downward displacement, the needle holding element 8 with the needle or the lancet 10 is likewise displaced depending on the configuration of the curved profile of a control curve section 13, which is preferably V-shaped and has a low point 13a.

In order to vary the orientation of the tensioning curve section 5, which is oriented at the angle 5a relative to a displacement movement direction or a longitudinal axis of the base body (main body) and is preferably slightly arc-shaped, it being designed in the manner of a web or rail, during the displacement movement of the manual actuating element and thus of the component 2, the base body (main body) 1 has a protrusion 14 as illustrated in the perspective plan view in FIG. 3. This protrusion 14 which is fixed to the base body (main body) causes a deflection or change in the course direction of the tensioning curve section 5 during the upward and downward movement of the component 2, wherein both the tensioning curve section and the control curve section and the plastic spring element 4 are arranged on a pivoting component 15 arranged pivotably on the component 2, as can be seen from FIGS. 4a, 4b and 4c. This pivoting component is preferably connected in one piece or alternatively in two pieces with the rest of the component 2.

In FIGS. 4a-4c, the component 2 is shown in individual assembly steps.

In FIG. 4a, the component 2 is composed both of the guide cutout 6 with the opening 7 at the lower side and also of the pivoting component 15, which at its lower side is preferably connected in one piece with the rest of the component 2 in an articulated manner, and of the plastic spring element 4.

The manual actuating element is preferably connected in one piece with the component 2.

In a first assembly step, the body of the component 2, which is preferably cast in one piece, is arranged together with the pivoting component 15 and then, as shown in FIG. 4b, the needle holding element 8 having the needle tip end 9 is inserted in the guide cutout 6.

In a third assembly step shown in FIG. 4c, the correct placement of the plastic spring element 4 then takes place, which can slide past one end 16 of the component 2 but is guided by the latter due to the bevelled shape of the end 16 during a pretensioning and pressure release phase.

The pivoting component 15 may be connected to the rest of the component 2 by means of a film hinge which is preferably plastic-based. The tensioning and control curve sections may be formed not only on the pivoting component 15 but also on the base body (main body) 1 and/or on the needle holding element 8, wherein the respective associated cams and groove guides are designed so as to be complementary to the components cooperating therewith. As an alternative to a plastic spring element, a steel spring may also be used, wherein such a steel spring may be mounted already in the pretensioned state in such a lancing device. This makes it possible to omit a pretensioning travel and the tensioning curve section.

FIG. 4c clearly shows the tensioning curve section 5 and the control curve section 13, along which there slides on the one hand a protrusion 14 fixed to the base body (main body) and on the other hand the needle holding element 8 which engages at the top and bottom in the control curve section 13, the latter likewise being designed as a web or rail.

FIGS. 5a-5e show a perspective view of various operating states of the lancing device according to the first embodiment of the invention.

FIG. 5a shows the lancing device before use, i.e. in the unused state. It comprises the base body (main body) 1 with the component 2 arranged therein and the needle exit 3 at the lower side and also the manual actuating element 12.

The tensioning curve section 5 is mounted so as to slide relative to the protrusion 14 fixed to the base body (main body), said protrusion having preferably a triangular basic shape.

As illustrated by the arrow 17, the entire component 2 can be pushed into the base body (main body) 1 by pressing the manual actuating element 12 from above.

FIG. 5b shows the operating state of pretensioning the plastic spring element 4. By pushing the manual actuating element 12 downwards into the base body (main body) 1, the control curve section 5 slides along the protrusion 14, as a result of which the plastic spring element 4 is pretensioned by being pushed to the left as a result of pivoting the pivoting part 15. This is illustrated by an enhanced arc shape of the plastic spring element 4 both in FIG. 5b and in FIG. 5c.

Such a pretensioning movement by displacing the component 2 in the direction of the lower end of the base body (main body) 1, as illustrated by the arrow 18, moreover causes the needle holding element 8 with two protruding cam elements or a groove arranged in the needle holding element to run along a web or a rail of the control curve section 13 from left to right. In the process, the needle holding element travels through the low point 13a of the control curve section 13, but at a point in time at which the component 2 has not yet been fully pushed into the base body (main body) 1 until it reaches the stop. As a result, the needle tip 9 is briefly pushed downwards and then upwards again due to the V-shaped profile of the control curve section, but does not yet exit from the lower end 3 of the base body (main body) 1.

As soon as the protrusion 14, which is designed in the manner of a cam, has reached a free end 5b of the control curve section 5—as illustrated in FIG. 5c as the start time of the pressure release—the tensioning curve section is released by the protrusion or cam 14, whereupon a pressure release of the curved spring element is triggered. This causes the pivoting part 15 to pivot back into its original state and causes a pressure release of the plastic spring element 4.

During such a pressure release, the needle holding element 8 once again travels along the control curve section 13, but this time in the opposite direction, whereupon, while travelling past the lowest point 13a of the control curve section 13, the needle 10 together with the tip end 9 and the needle holding element 8 are briefly moved forwards. This takes place to such an extent that the tip end 9 is briefly moved out of the opening 3 of the base body (main body) 1 and then is pushed back in again since the needle holding element 8 then travels along the second portion of the control curve section 13, namely the left-hand portion. Such a lancing movement is therefore driven by a spring and is linear in terms of its movement course, resulting in an almost user-independent lancing movement and thus in an almost neutral movement of the needle with regard to the pain caused. The linear lancing movement is illustrated by the arrow 19.

FIG. 5e shows the lancing device after use. In this operating state, the needle tip is retracted back into the base body (main body) 1 along with the needle holding element 8, as illustrated by the arrow 20.

The component 2 on the other hand remains arranged within the base body (main body) 1 and no longer moves back. After such a single use, such a lancing device is not intended for further use.

FIG. 6a shows a perspective view of a lancing device according to a second embodiment of the invention. This lancing device once again comprises a base body (main body) 21 together with a component 22 and an end 23 which is open at the bottom and which may be designed in the manner of a hole.

FIG. 6b shows a perspective view of the lancing device according to the second embodiment of the invention in the disassembled state. The component 22 once again comprises the plastic spring element 24 and the tensioning curve section 25. Also provided is the guide cutout 25 with a lower end 27 for receiving the needle holding element 28.

The needle holding element 28 once again comprises a needle with a tip end 29, which is covered by means of a protective cap 31.

FIGS. 7a and 7b show an open and partially cross-sectional front view of the lancing devices according to the second and first embodiments for comparison purposes.

The lancing device according to the second embodiment of the invention, which is shown in FIG. 7a, shows the component 22 with the plastic spring element 24 and the tensioning curve section 25, which once again is arranged at an angle 25a to the direction of displacement of a manual actuating element 32 and thus of the component 22 and also to the displacement movement of the needle 30 together with the needle holding element 28. Once again, the tensioning curve section 25 may be slightly arc-shaped and may be made from inelastic or elastic material with regard to its arc deformability. The tensioning curve section 25 may likewise be designed either as a groove with one or more cams of complementary shape engaging therein, said cam or cams being arranged on the base body (main body) 1, or as a web or rail along which a cam fixed to the base body (main body) can slide.

The tensioning curve section 25 once again comprises a free end 25b.

A control curve section 33 is once again V-shaped in an identical or similar manner and has a lowest control curve section point 33a at the bottom.

Unlike the lancing device according to the first embodiment which is shown in FIG. 7b, in the lancing device according to the second embodiment shown in FIG. 7a the return movement of the pivoting part 15 brings about a spring excursion at the same time as the end position of the component 22 and of the actuating element 32 is reached. This is achieved by means of a fixed stop within the housing.

By contrast, in the lancing device according to the first embodiment of the invention in FIG. 7b, a return movement of the pivotable part is obtained already before the component 2 or the manual actuating element 12 has reached its end position, i.e. before the component 2 has been fully pushed into the base body (main body).

As a result, the residual travel of the manual actuating element 12 or of the component 2 within the base body (main body) 1 is superposed on the pivoting movement of the pivoting part 15, which also performs the lancing stroke. In this case, the protrusion 14 fixed to the housing is configured in such a way that on the one hand the spring pressure further assists the linear displacement of the component 2 and on the other hand the pivotable part 15 does not reach the maximum lancet exit extent or tip end exit extent until the component 2 has reached its end stop within the base body (main body). This is important in terms of the repetition precision of the lancing depth.

Advantageously, the lancing device according to the first embodiment of the invention has a smaller overall size since the pivotable part 15 has to travel through a smaller angle and thus can also be of a more robust configuration since the plastic spring element has to be tensioned to a lesser degree.

FIGS. 8a-8c show a perspective view of the lancing device according to the second embodiment of the invention in various assembly steps. Once again, the component 22 has a pivotable part 35 which is suspended in a pivotable manner at the lower side, preferably by means of a film hinge.

Then, in a second step shown in FIG. 8b, the needle holding element 28 is inserted.

In a third step, the pivoting part 35 and the rest of the component 22 are pivoted together in such a way that the plastic spring element 24 can slide past an underside of the component 22, the underside 36 being slightly bevelled so that it can slide past with little friction.

The control curve section 33 is once again V-shaped. The tensioning curve section 25 is located below the control curve section 33.

FIGS. 9a-9e show perspective views of the lancing device according to the second embodiment of the invention during the operation thereof.

FIG. 9a shows the operating state before use of the lancing device. The rear side of the component 22 is still located together with the manual actuating element 32 somewhat outside the base body (main body) 21 and is pushed into the base body (main body) 21 by a manual application of pressure, as illustrated by the arrow 37.

FIG. 9b shows a perspective view of the component 22 being pushed into the base body (main body) 21, as illustrated by the arrow 38. During this pushing-in, a pretensioning of the plastic spring element 24 takes place by pivoting the pivoting part 35 to the left. This takes place as a result of the tensioning curve section 25 sliding along the protrusion 34 fixed to the base body (main body), which protrusion may once again preferably be triangular.

Upon reaching a free end 25b of the tensioning curve section 25, the pressure in the plastic spring 24, which is pretensioned to its maximum, is suddenly released, as illustrated in FIG. 9c, so that a pressure release of the plastic spring element 24 is started. This takes place as a result of the fact that the protrusion 34 releases the tensioning curve section 25 by exceeding the free end 25b of the tensioning curve section 25.

During the pressure release, the needle holding element 28 is pushed forwards within the guide cutout 26, since it travels along the V-shaped control curve section from right to left within a very short period of time and briefly reaches the lowest point 33a of the control curve section 33. As it reaches this point, the end 29 of the needle 30 briefly exits from the opening 23 at the lower side of the base body (main body) 21 and is then immediately retracted back into the base body (main body), as illustrated by the arrow 40 in FIG. 9e. FIG. 9e shows the state of the lancing device as a whole according to the second embodiment of the invention after use.

Of course, the needle holding element with a groove arranged therein or two protrusions arranged thereon already travels along the control curve section, which is designed in the manner of a web or rail, from left to right a first time during the downward pushing of the actuating element 32, before it travels along the control curve section a second time in the opposite direction in order during this second time to allow the needle to be pushed out of the base body (main body), unlike the first time.

FIG. 10a shows a perspective view of a lancing device according to a third embodiment of the invention. This lancing device once again comprises a base body (main body) 41 with a component 42 arranged therein and an opening 43 at the lower side.

In FIG. 10b, the lancing device according to the third embodiment is shown in the disassembled state. This lancing device comprises on its component a plurality of elements preferably formed in one piece therewith, which comprise the plastic spring element 44, a pivotable part 45 which at the top is pivotably arranged on the component and has a control curve section at the bottom, and an actuating element 52.

Once again, a guide cutout 46 is arranged in the component 42 in order to arrange a needle holding element 48 with a needle 50 arranged therein, said needle having a tip end 49 with a protective cap 51 arranged thereon.

The protective cap 51 can be twisted off in a separate operating step prior to actuation of the lancing aid and serves as sterile protection.

FIG. 11 once again shows the component 42 of the lancing device according to the third embodiment. The component 42 comprises the pivotable part 45, which is pivotably arranged at the top by means of a film hinge 55. At the bottom, the pivotable part is provided with a control curve section 53, which is once again V-shaped in an identical or similar manner and has a lowest point 53a.

The plastic spring element 44 can be pivoted together with the manual actuating element 52, which is pivotably arranged at a lower point 54 by means of a film hinge on the rest of the component 42, relative to the rest of the component 42, as illustrated by the double-headed arrow 52.

The pivotable manual actuating element 52 can latch with its upper end 52a into a corresponding corner edge 42a of complementary shape on the component 42, as soon as the manual actuating element has been pivoted far enough into the base body (main body).

FIG. 12 once again shows an open and partially cross-sectional front view of the lancing device according to the third embodiment of the invention. It can clearly be seen from this diagram that the plastic spring element 44 can be mounted relative to a stop 56 of the pivotable part 45 in order to bring about a release of the needle holding element upon further actuation of the manual actuating element 52.

By means of a groove, the needle holding element 48 can slide along the control curve section 53 while the control curve section 53, which is designed in the manner of a rail or web, moves from left to right.

FIGS. 13a-d once again show a perspective view of various operating states of the lancing device according to the third embodiment of the invention. FIG. 13a shows the lancing device before use. At the start, the manual actuating element 52, which is arranged at the side, is pivoted to the right as illustrated by the arrow 57.

By virtue of such a pivoting of the manual actuating element 52 to the right, a pretensioning of the plastic spring element 44 takes place—as shown in FIG. 13b—by the latter being pushed with its right-hand end (not shown here) against the stop 56 (shown in FIG. 12) of the pivotable part 45. As a result, an enhanced arc shape of the plastic spring element 44 is obtained and thus a pretension is built up. This is illustrated by the arrow 58.

In FIG. 13c, an unlocking of the arrested state of the needle holding element 48 takes place through the actuation of an arresting lever 48a, which is shown in more detail in FIG. 11 and is connected to the needle holding element 48, by means of a protrusion 59c of the manual actuating element 52, as a result of which the pressure of the pretensioned plastic spring 44 can suddenly be released and the pivotable part 45 is suddenly pivoted to the right, as illustrated by the arrows 59 and 60 in FIGS. 13c and 13d.

Such a pivoting of the pivotable part 45, in particular of the lower end thereof, makes it possible for the needle holding element 48 to be displaced from right to left along the control curve section 53, which is arranged at the lower end of the pivotable part 45, whereupon the needle tip 49 briefly exits from the lower end 43 and 47 of the base body (main body) and of the component 42 and is immediately retracted back into the lancing device after the lowest point 53a of the control curve section 53 has been passed. As a result, a user-independent, quick and easy lancing operation takes place.

If the plastic spring element 44 is fully pretensioned, such a pivoting movement of the pivotable part 45 in a controlled manner is possible at the same time as the upper end 52a of the manual actuating element 52 latches into the corner section 42a of the component 42, as illustrated by the diagram shown in FIG. 13c.

FIG. 14 once again shows an open and partially cross-sectional front view of a lancing device according to a fourth embodiment of the invention. By comparing this with a component 62 which is shown in perspective view in FIG. 15 and which is arranged within a base body (main body) 61 with an opening 63 at the lower side, it is clear that once again a plastic spring element 64 of arc-shaped design is arranged on a manual actuating element 72, wherein in this case the manual actuating element and the plastic spring element 64 are at an angle of almost 90° relative to one another.

The plastic spring element can be pushed against a stop 76 of a pivotable part 65 in the direction of a double-headed arrow 72b, by pushing the manual actuating lever 72 downwards from above.

Once again, a latching mechanism is provided between the component 62 with a protrusion 62a and a protrusion part 65a of the pivotable part 65 which acts like a spring or is of elastic design. This elastic part 65a can be released by a protrusion 72b of the manual actuating element 72 in order to trigger a pressure release once the plastic spring element 64 has been pretensioned.

The needle holding element 68 once again comprises the needle 70 with a tip end 69.

The pivotable element 65 is arranged such that it can be pivoted relative to the rest of the component 62 at its lower end preferably by means of a film hinge 75.

Likewise, the manual actuating element 72 is arranged such that it can be pivoted relative to the rest of the component 62 by means of a film hinge 74.

The component 62 is once again equipped with a guide cutout 66 which has a lower open end 67.

The pivotable part 65 this time has at the top a V-shaped control curve section 73, which once again includes a lowest point 73a.

FIGS. 16a-d show a perspective, partially open view of the lancing device according to the fourth embodiment of the invention in various operating states. FIG. 16a shows the operating state before use. In this state, the control curve section 73 of the pivotable part 65 is still arranged with its right-hand end relative to the needle holding element 68.

As soon as a pivoting movement of the manual actuating element 72 in the downward direction from above takes place, as illustrated by the arrow 77, a pretensioning movement of the plastic spring element 64 takes place, without the pivotable part 65 being pivoted, since the latter is still arrested by means of the latching mechanism 65a, 62a. This is illustrated in FIG. 16b, wherein the pivoting movement is shown by the arrow 78.

As soon as the protrusion 72a of the manual actuating lever pushes against the elastic protrusion 75a of the latching mechanism, the latter is moved out of the latching stop 62a of the component 62 and thus the pivotable part 65 is released for a pivoting movement from left to right, as illustrated by the arrow 79 in FIG. 16c. As a result, the needle holding element 68 can run along the control curve section 73 from right to left or the control curve section 73 moves through the needle holding element 68 from left to right. As a result, a brief excursion and then a retraction of the needle tip 69 out of the bottom opening 63 takes place. This is illustrated in FIG. 16c.

FIG. 16d shows the lancing device after use. In this state, the pivotable part 65 has been fully pivoted from left to right, as illustrated by the arrow 80.

The pivoting elements may be designed in such a way that, instead of being arranged pivotably on the base body (main body) by means of film hinges and thus being formed in one piece therewith, they are provided as displaceable elements which can be displaced relative to the base body (main body).

All of the features disclosed in the application documents are claimed as essential to the invention in so far as they are novel individually or in combination with respect to the prior art.

### LIST OF REFERENCES

1 base body (main body)

2 component

3 needle exit

4 plastic spring element

5 tensioning curve section

5a angle

5b free end

6 guide cutout

7 opening on the lower side

8 needle holding element

9 tip end

10 needle/lancet

11 protective cap

12 manual actuating element

13 control curve section

13a low point of the profile

14 protrusion

15 pivoting component

16 end

17 arrow

18 displacement movement

19 displacement movement

20 displacement movement

21 base body (main body)

22 component

24 plastic spring element

25 tensioning curve section

25a angle

25b free end

26 guide cutout

28 needle holding element

29 tip end

30 needle

31 protective cap

32 manual actuating element

33 control curve section

33a profile

34 protrusion

35 pivoting part

36 underside

37 arrow

38 displacement movement

39 displacement movement

40 displacement movement

41 base body (main body)

42 component

42a latching mechanism/corner edge

43 opening

44 plastic spring element

45 pivoting element

46 guide cutout

48 needle holding element

48a retaining protrusion/arresting lever

49 tip end

50 needle

51 protective cap

52 double-headed arrow

52a upper end of the latching mechanism

52c release protrusion

53 control curve section

53a profile

55 film hinge

56 stop

57, 58, 59 arrow

59c protrusion

60 arrow

61 base body (main body)

62 component

62a latching mechanism

63 opening on the lower side

64 plastic spring element

65 pivoting element

65a retaining protrusion

66 guide cutout

67 open end

68 needle holding element

69 tip end

70 needle

72 manual actuating element

72a release protrusion

72b protrusion

73 control curve section

73a profile/lowest point

75 film hinge

75a elastic protrusion

77, 78, 79, 80 arrow

1. A lancing device for taking blood for medical examinations, comprising a base body, at least one needle which is arranged therein and which can be extended outwards with a tip end, having a needle holder which at least partially encompasses the needle, and a manual actuator for releasing a displacement movement of the needle together with the needle holder relative to the base body, wherein a movable plastic spring for generating a pretension of the manual actuator, which is connected to a control curve section that is configured to move within the base body, is in at least one component arranged in the base body, wherein the needle holder is connected to the control curve section and is configured to move in the forward and backward directions during a pressure release of the plastic spring by travelling along the control curve section.

2. The lancing device according to claim 1, comprising a tensioning curve section which is arranged on the component and runs at an angle to the direction of the displacement movement of the needle holder, which tensioning curve section is connected to the plastic spring, of arc-shaped design, and, during a displacement movement of the component relative to the base body, can be varied in terms of its course direction.

3. The lancing device according to claim 2, wherein the displacement movements of the outwardly moving holder and of the component have the same direction.

4. The lancing device according to claim 2, wherein the displacement movements of the outwardly moving needle holder and of the component run at an angle to one another.

5. The lancing device according to claim 16, wherein the protrusion has a triangular shape.

6. The lancing device according to claim 2, wherein the tensioning curve section has a free end which can automatically be guided into its starting position for the pressure release of the plastic spring.

7. The lancing device according to claim 2, wherein the component is formed in one piece together with the control curve section, the tensioning curve section and the plastic spring.

8. The lancing device according to claim 1, wherein the plastic spring is arranged on the manual actuator, which is movable relative to the component, and can be pretensioned against a pivoting element which comprises the control curve section and is mounted such that it can pivot relative to the component.

9. The lancing device according to claim 8, wherein the manual actuator has a release protrusion for displacing a retaining protrusion out of an arrested position.

10. The lancing device according to claim 1, further comprising a latching mechanism for latching the manual actuator in a predefined position.

11. The lancing device according to claim 8, wherein the component and the pivoting element are formed in one piece.

12. The lancing device according to claim 1, wherein the component and the manual actuator are formed in one piece.

13. The lancing device according to claim 1, wherein the control curve section has a profile identical or similar to a V-shape.

14. The lancing device according to claim 1, wherein the control curve section forms in its profile a web which is encompassed on both sides by the needle holder so as to slide along it in the web profile direction.

15. The lancing device according to claim 1, wherein the control curve section forms in its profile a groove-like depression in which there engages at least one protrusion arranged on the needle holder, so as to slide along therein in the depression profile direction.

16. The lancing device of claim 2, wherein the tensioning curve section connected to the plastic spring can be varied in terms of its course direction by sliding along a protrusion arranged on the base body during a displacement movement of the component relative to the base body.

17. The lancing device of claim 2, wherein the tensioning curve section connected to the plastic spring can be varied in terms of its course direction by sliding along a provided groove-like element during a displacement movement of the component relative to the base body.

18. The lancing device of claim 10, wherein the latching mechanism for latching the manual actuator in a predefined position is connectively associated with the manual actuator and the component.

19. The lancing device according to claim 1, wherein the tensioning curve section forms in its profile a groove-like depression in which there engages at least one protrusion arranged on the needle holder, so as to slide along therein in the depression profile direction.

20. The lancing device according to claim 1, wherein the plastic spring is moveable in at least one of a displacement or pivoting fashion.

21. The lancing device according to claim 1, wherein the control curve section is configured for movement in at least one of a displacement or pivoting fashion.
